(19) United States
(12) Patent Application Publication
(10) Pub. No.: US 2012/0330163 A1
(43) Pub. Date: Dec. 27, 2012

(54) MEASURING APPARATUS

(75) Inventor: Takaaki Nakabayashi, Kawasaki-shi (JP)

(73) Assignee: CANON KABUSHIKI KAISHA, Tokyo (JP)

(21) Appl. No.: 13/582,021

(22) PCT Filed: Apr. 20, 2011
(86) PCT No.: PCT/JP2011/002312

§ 371 Date: Aug. 30, 2012

(30) Foreign Application Priority Data
Apr. 22, 2010 (JP) 2010-098712
Mar. 9, 2011 (JP) 2011-052069

(51) Int. Cl. A61B 6/04

(52) U.S. Cl. 600/476

(57) ABSTRACT

The present invention provides a measuring apparatus including: a supporting member which supports a subject person and has an opening for inserting an object that is a part of the subject person; and a first holding member and a second holding member for interposing and compressing the object when the object is inserted through the opening, wherein at least two of the supporting member, the first holding member and the second holding member can move independently from one another in a direction of compressing the object.

### TECHNICAL FIELD

The present invention relates to a measuring apparatus having a supporting member for supporting a subject person so that an object is compressed and held.

### BACKGROUND ART

As a conventional apparatus for obtaining biological information, an X-ray diagnostic apparatus, an ultrasonic diagnostic apparatus and a measuring apparatus using a photoacoustic effect are known. Biological information obtained by such an apparatus is processed into an image and is made us of for diagnosis. In order to obtain a good measurement result using these apparatuses, a measuring area must be held so that an object does not move. On the other hand, if the object is human biological tissue to be diagnosed, it is also required that the burden of measurement must be minimized to avoid discomfort.

The photoacoustic effect is a phenomena where a pulsed light is irradiated from a light source, such as a laser, onto an object, and the object which absorbs the light energy and expands/contracts, and generates an acoustic wave (photoacoustic wave). By detecting the photoacoustic wave using a probe, processing the signals and reconstructing an image, the optical characteristic value distribution inside the object can be obtained and visualized.

Japanese Patent Application Laid-Open No. H07-303633 (PTL 1) discloses an X-ray diagnostic apparatus which has a bed for decreasing the burden on a subject person, and compressing means for securing the projected sectional area of a breast that is sufficient for capturing the image.

FIG. 9 is a diagram depicting an X-ray mammography apparatus disclosed in Japanese Patent Application Laid-Open No. H07-303633. This apparatus has a bed 113 where a breast insertion opening is created, a compression plate 102 for compressing the breast 112, and an X-ray film table 101. The X-ray film table 101 is disposed such that the breast 112 can be interposed with the compression plate 102, and encloses an X-ray film 105. During the mammography, a subject person lies on her stomach on the bed and inserts their breast 112 into the breast insertion opening. The inserted breast 112 is located between the compression plate 102 and the X-ray film table 101. In this state, the breast 112 is compressed by moving the compression plate 102. Then an X-ray beam is irradiated from an X-ray irradiation unit 117 onto the breast 112 so as to capture the image.

In a case of a breast being compressed vertically with two compression plates while the subject person is standing or sitting, an operator must compress the breast while holding it or pressing down on it by hand in order to secure a projected sectional area of the breast that is sufficient for mammography. According to the configuration of Japanese Patent Application Laid-Open No. H07-303633, the subject person lies on her stomach on a bed, so the breast vertically hangs down, and the projected sectional area increases for the amount hanging down. Another effect is that an image of the breast can be captured in a confortable position without giving pain to the subject person.

### CITATION LIST

### Patent Literature

### [PTL 1]

Japanese Patent Application Laid-Open No. H07-303633

### SUMMARY OF INVENTION

### Technical Problem

In the case of the X-ray mammography apparatus disclosed in Japanese Patent Application Laid-Open No. H07-303633, the positions of the bed 113 and the X-ray film table 101 are fixed with respect to the compressing direction. Hence if the subject person lies down on her stomach on the bed 113 and her breast 112 is inserted into the space between the compression plate 102 and the X-ray film table 101, the lower part of the breast may be separated from the X-ray film table 101. In order to obtain a projected sectional area of the breast that is effective for capturing the image, an upper portion of the breast or a lower portion of the breast and the compression plate are contacted for positioning in advance, then the breast is compressed between the compression plates at the contacting side and the opposite side. For the positioning between the lower portion of the breast and the X-ray film table 101, the subject person must move to adjust their position, since the apparatus does not have a positioning unit, so the burden is on the subject person.

FIG. 10 shows the states of a breast which is compressed without positioning. FIG. 10A is a diagram depicting a case when the compression plate 102 is moved and contacted with the upper portion of the breast. A gap between the breast 112 and the X-ray film table 101 is seen in the lower portion of the breast. If the compression plate 102 is further moved in this state, the breast 112 is shifted toward the bed 113 along with the movement of the compression plate 102, since the breast 112 is not compressed and held between the compression plate 102 and the X-ray film table 101. FIG. 10B is a diagram depicting a case when the breast is compressed and held. As FIG. 10B shows, in the state of the breast being compressed and held, the breast 112 moves up compared with the state before being compressed and held, and the projected sectional area H decreases.

The above problem is common to apparatuses which measure biological information with securing a breast, and the same is true for an ultrasonic diagnostic apparatus and a photoacoustic measuring apparatus, in addition to an X-ray diagnostic apparatus. Particularly in a case of the photoacoustic measuring apparatus, it is preferable to fix the compression plate having a probe and an optical system in a housing, just like the above mentioned X-ray film table, if possible, in order to guarantee the positional accuracy of the probe and the optical system. In this case, it is further demanded to perform positioning of the fixed compression plate and the breast accurately.

With the foregoing in view, it is an object of the present invention to provide a technology for simply positioning an object in a measuring apparatus while decreasing the burden on the subject person.

### Solution to Problem

This invention provides a measuring apparatus comprising:
a supporting member which supports a subject person and has an opening for inserting an object that is a part of the subject person; and a first holding member and a second holding member which interpose and compress the object when the object is inserted through the opening, wherein
at least two of the supporting member, the first holding member and the second holding member can move independently from one another in a direction of compressing the object.

### Advantageous Effects of Invention

According to the present invention, an object can be easily positioned in a measuring apparatus while decreasing the burden on the subject person.

Further features of the present invention will become apparent from the following description of exemplary embodiments with reference to the attached drawings.

### BRIEF DESCRIPTION OF DRAWINGS

FIG. 1 is a perspective view of a measuring apparatus according to Example 1.

FIG. 2 is a side view of inside the measuring apparatus according to Example 1.

FIG. 3 is a perspective view of a holding mechanism according to Example 1.

FIG. 4 is a side view depicting a probe unit, a projection unit and a light source according to Example 1.

FIG. 5 are schematics depicting how to hold a breast according to Example 1.

FIG. 6 is a side view of a supporting member and holding mechanism of a measuring apparatus according to Example 2.

FIG. 7 are schematics depicting how to hold a breast according to Example 2.

FIG. 8 are schematics of a variant form of Example 2.

FIG. 9 shows a configuration of a conventional X-ray mammography apparatus.

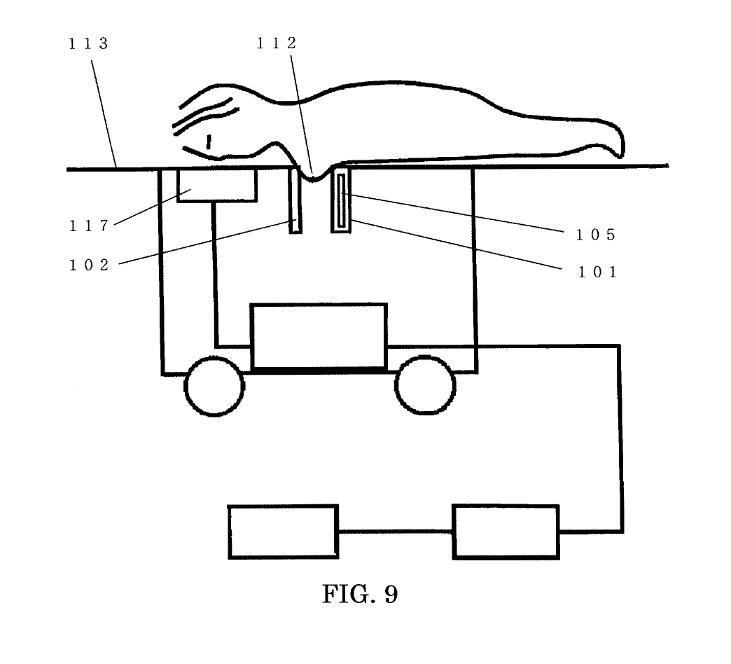

FIG. 10 are schematics depicting a case of compressing a breast without positioning according to a conventional configuration.

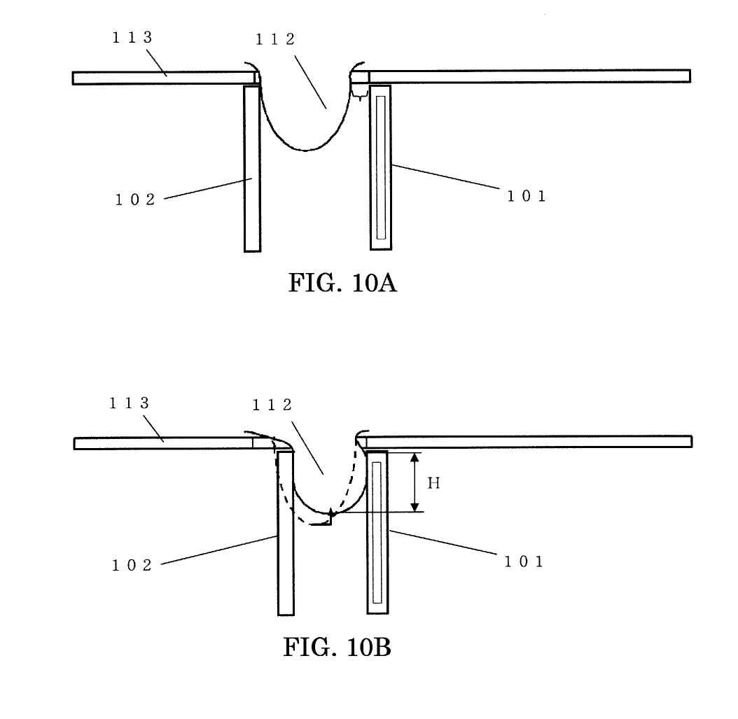

### DESCRIPTION OF EMBODIMENTS

Preferred embodiments of the present invention will now be described with reference to the drawings. Measuring apparatuses in the following examples are measuring apparatuses for obtaining image data on a breast of a subject person, which is an object, using the photoacoustic effect. In other words, an object is the breast, which is a part of the subject person. For this, a photoacoustic wave (e.g. ultrasound) generated from the object which has absorbed light energy of a laser beam or the like irradiated from a light source, is detected. Dimensions, materials and shapes of components described hereinbelow and relative disposition thereof, however, should be appropriately changed depending on the configuration of an apparatus to which the present invention is applied and various conditions, and are not for limiting the scope of the present invention to the following description. For example, the method for securing a breast using the apparatus of the present invention can also be applied to a mammography apparatus using X-rays or ultrasonic echoes.

In other words, the measuring apparatus of the present invention can be applied to any measuring apparatus, if the apparatus includes a supporting member which supports a subject person and has an opening for inserting an object that is a part of the subject person, and a first holding member and a second holding member for interposing and compressing the object when the object is inserted through the opening. Characteristics of the present invention is that at least two of the supporting member, the first holding member and the second holding member can move independently from one another in a direction of compressing the object.

### Example 1

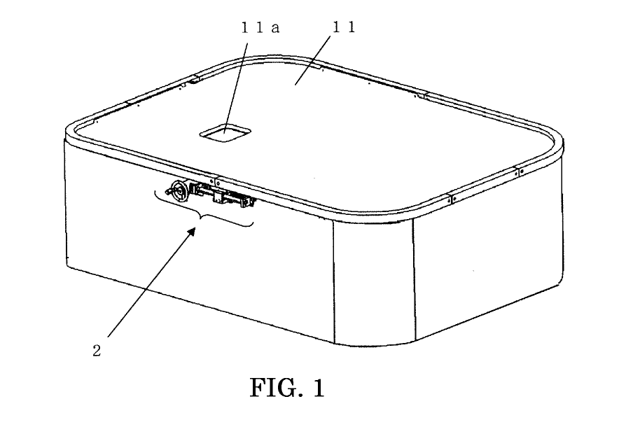

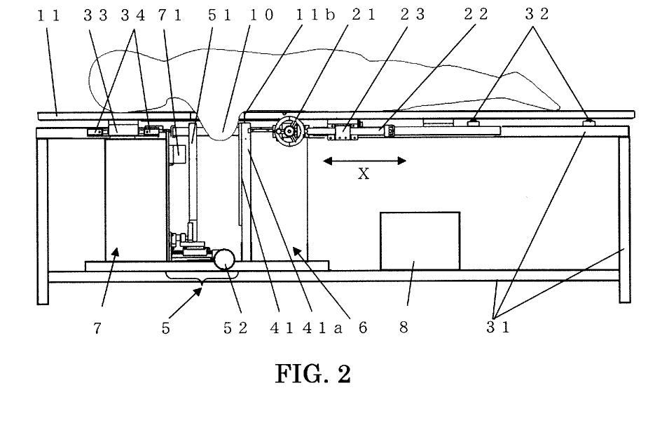

FIG. 1 is a perspective view of a measuring apparatus according to Example 1 of the present invention. FIG. 2 is a side view depicting inside the apparatus. In a bed 11 which is a supporting member for supporting a subject person, an opening 11a to insert a breast 10 is formed. A slide mechanism 2 is installed on the bed for sliding the bed in the X direction, shown in FIG. 2. By this slide mechanism 2, the breast 10 with the entire bed 11 can move relatively with respect to a later mentioned fixed holding plate 41 and a movable holding plate 51.

The slide mechanism 2 is comprised of a bed handle 21 for adjusting a position of the bed 11, a lead screw 22, and a lead screw nut 23 which interfits with the lead screw 22. The lead screw nut 23 is fixed to the bed 11. A frame 31 supports the bed as a housing. Rollers 32 are disposed on an upper surface of the frame 31. The bed handle 21 and a linear guide 33 are disposed on a side surface of the frame 31. By this configuration, the bed, which is the supporting member, can slide independently from the housing.

The bed 11 is installed on the rollers 32, and interfits with the linear guide 33, so as to slide only in the X direction. A stopper 34, for controlling a moving distance of the bed 11, is disposed so that an edge 11b of the opening 11a of the bed 11 does not move to the feet side from a mounting plate 41a of the later mentioned fixed holding plate 41. This prevents the breast 10 from being caught between the edge 11b of the opening 11a and the mounting plate 41a when the bed 11 is slid for adjustment. A stopper 34 is also disposed on the head side so as to control the moving distance of the bed 11.

Because of a self-lock mechanism of the lead screw 22, the bed 11 does not move even if a force is directly applied to the bed 11 in the X direction. In other words, the bed 11 is always locked unless the bed handle 21 is operated. It is preferable that the position adjustment mechanism of the bed is locked except during adjustment, and for example, the bed may slide without using the lead screw, so that the position is locked using a lock mechanism after adjusting the position. A motor may be used for the driving.

A fixed holding plate 41, a holding mechanism 5, a probe unit 6 and a projection unit 7 are disposed in a frame 31. The fixed holding plate 41 is a first holding member for compressing and holding the breast. A preferable material of the fixed holding plate 41 is polymethyl pentene for example, since an acoustic wave and light do not attenuate much. The movable holding plate 51 is a second is a second holding member disposed facing the fixed holding plate 41, and the position thereof is adjusted by a compression handle 52.

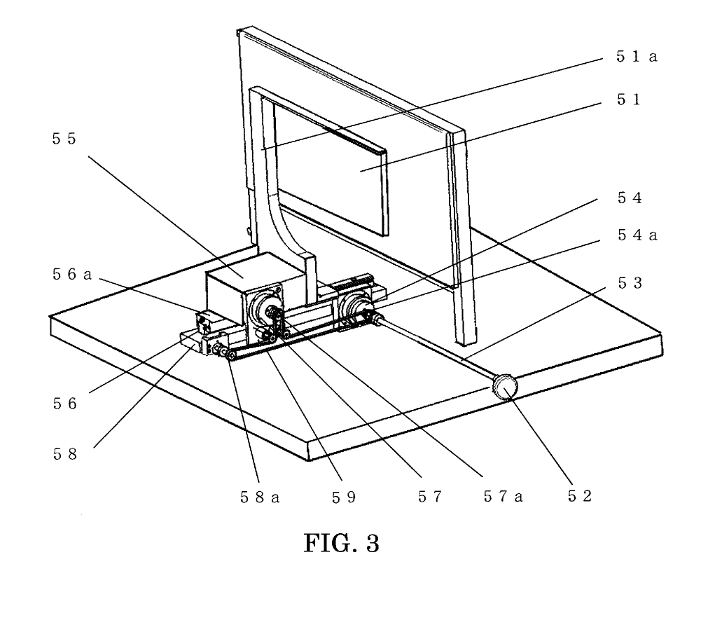

FIG. 3 is a perspective view depicting the holding mechanism 5. The compression handle 52 is connected with a transmission shaft 53. The other end of the transmission shaft 53 is connected with a first electromagnetic brake 54. The first electromagnetic brake 54 is electrically coupled with a switch, which is not illustrated, so that ON or OFF can be selected for the brake by operating the switch. A sprocket 54a is disposed in the first electromagnetic brake 54. If the first electromagnetic brake 54 is ON, rotation of the sprocket 54a is locked, and if OFF, the lock is released.

The movable holding plate 51 is fixed to a holding plate base 51a, and the holding plate base 51a is connected with a carrier 56a of a holding plate linear guide 56 via a connecting member 55. The holding plate linear guide 56 is disposed so as to match with the moving direction of the bed 11 (X direction in FIG. 2) by the slide mechanism 2. By this configuration, the bed 11, the fixed holding plate 41 and the movable holding plate 51 relatively move in a direction of compressing the breast 10. Since the breast 10 moves together with the bed 11, the breast 10 is positioned with respect to the fixed holding plate 41 and the movable holding plate 51 is moved, whereby the breast 10 is compressed and held.

In this example, the sliding direction of the bed 11 and the moving direction of the movable holding plate 51 match, but the moving direction is not limited to the linear movement, but may be rotary movement, or may be a combination of the linear movement and the rotary movement. A second electromagnetic brake 57 having a sprocket 57a is also connected with the connecting member 55. Just like the first electromagnetic brake 54, the second electromagnetic brake 57 is also electrically coupled with a switch, which is not illustrated, and ON or OFF can be selected for the brake by operating the switch.

The holding plate linear guide 56 is installed on a linear guide base 58, and the linear guide base 58 has a sprocket 58a. A chain 59 is engaged with the sprockets 54a, 57a and 58a. Rotary driving by the compression handle 52 is transferred to the connecting member 55 via the chain 59, sprockets 54a, 57a and 58a, and the connecting member 55 moves along the linear guide 56, whereby the movable holding plate 51 moves. The holding mechanism 5 compresses the breast 10 by manual compression using the compression handle 52, but may be driven by a motor. A torque limiter may be used so that excessive force is not applied to the breast 10.

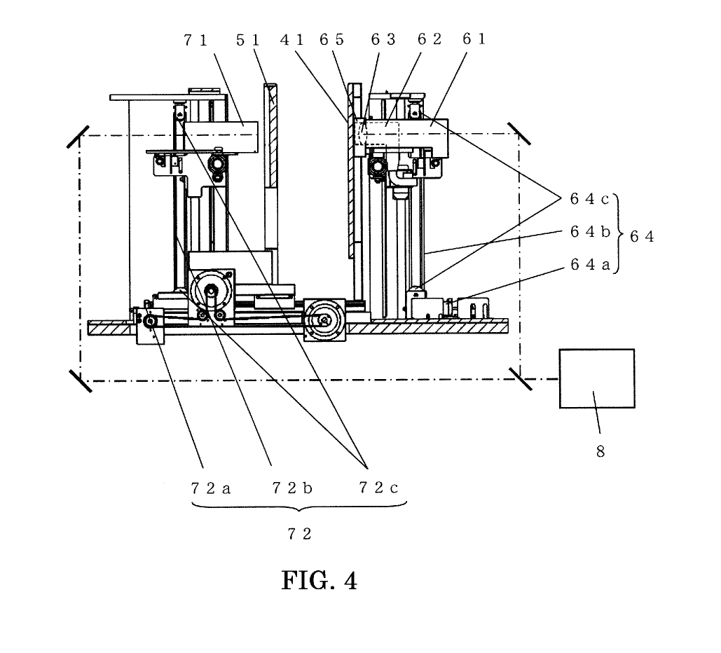

FIG. 4 is a cross-sectional side view depicting the probe unit 6, the projection unit 7 and the light source 8. A probe carriage 61 includes a probe 62 for receiving an acoustic wave and an optical system 63 for irradiating the light guided from the light source 8 to the probe carriage 61 onto the breast 10. The breast 10 is two-dimensionally scanned in parallel with the fixed holding plate 41 using a probe scanning mechanism 64. The probe scanning mechanism 64 is comprised of a motor 64a, a belt 64b and a pulley 64c (a scanning mechanism in the direction perpendicular to the page surface is not illustrated). An oil seal 65 is disposed on the probe carriage 61 so as to contact with the fixed holding plate 41. Matching oil, which is not illustrated, fills a space between the probe 62 and the fixed holding plate 41.

The projection unit 7 is comprised of a projection carriage 71 where an optical system for irradiating light from the movable holding plate 51 side is disposed, and a projection scanning mechanism 72 which scans the projection carriage 71 to an opposite position according to scanning of the probe carriage 61. The projection scanning mechanism 72 is comprised of a motor 72a, a belt 72b and a pulley 72c (a scanning mechanism in the direction perpendicular to the page surface is not illustrated). Therefore a material in which light is not attenuated much is preferable for the movable holding plate.

According to the configuration of this example, the fixed holding plate 41 and the probe unit 6 are integrated in a positional relationship, therefore contact of the oil seal 65 and the fixed holding plate 41 can be maintained. Furthermore, the positional accuracy between the light guided to the probe carriage 61 and the optical system 63 can be maintained by installing the fixed holding plate 41 and the probe unit 6 in the frame 31, so as to fix the positions thereof with respect to the light source 8.

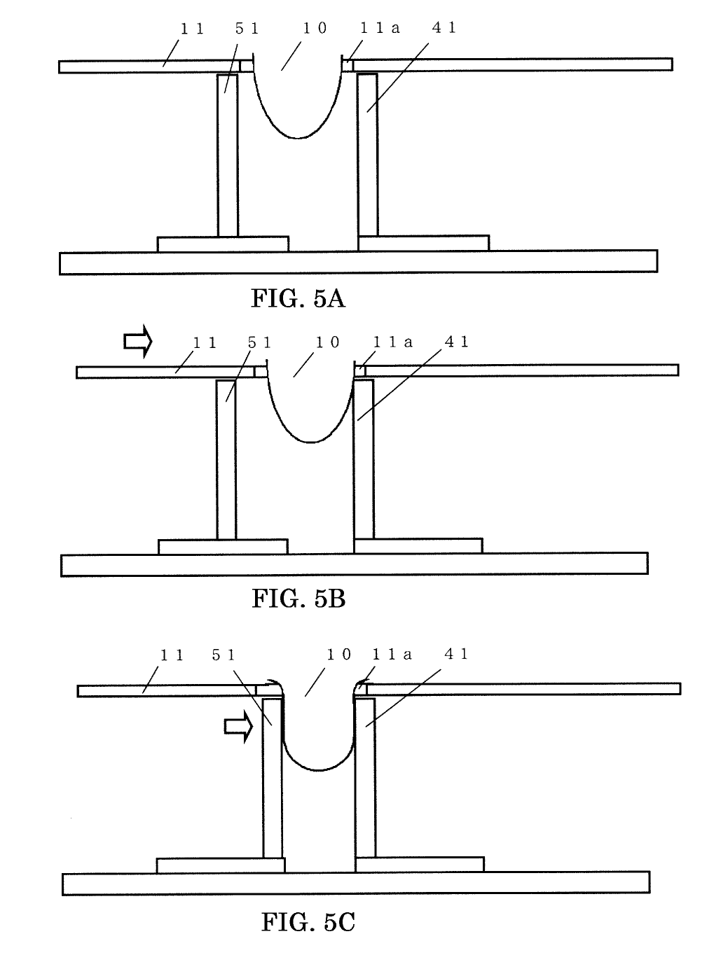

Now positioning between the breast 10 and the fixed holding plate 41 in the case of obtaining an acoustic wave generated from the breast 10 will be described. FIG. 5 are schematics depicting the positional relationships of the breast 10, the bed 11, the fixed holding plate 41 and the movable holding plate 51 from inserting the breast into the opening 11a to compressing and holding the breast 10, and the frame 31 and other components are omitted. FIG. 5A shows a state when the breast 10 is inserted into the opening 11a. FIG. 5B shows a state when a lower portion of the breast is contacted with the fixed holding plate 41. FIG. 5C shows a state when an upper portion of the breast is compressed and held using the movable holding plate 51.

The subject person first lies on her stomach on the bed 11, and inserts her breast 10 of which image is to be obtained into the opening 11a (FIG. 5A). Then the bed handle 21 is rotated to the slide th bed 11. Here the bed 11 is moved to a position where a lower portion of the breast 10 contacts the fixed holding plate 41 (FIG. 5B). Then the compression handle is rotated to move the movable holding plate 51 independently (FIG. 5C). In this case, the electromagnetic brake 54 is OFF, and the sprocket 54a is not locked. The electromagnetic brake 57 is ON, and the sprocket 57a is locked. Therefore the movable holding plate 51 moves to the fixed holding plate 41 side by rotating the compression handle 52. If the breast 10 is compressed for a required compression amount, the switch of the electromagnetic brake 54 is turned ON to fix the position of the movable holding plate 51 and to hold the breast 10.

Then light is irradiated from the light source 8 onto the object. Due to this, light is irradiated from the optical system 63 on the probe unit side and the projection carriage 71 onto the breast 10. Then the probe 62 receives an acoustic wave (photoacoustic wave) emitted from the breast 10, and converts the acoustic wave into an electric signal. The electric signal obtained by the probe 62 is sent to a signal processor, which is not illustrated, and is used for reconstructing an image, and image data is thus regenerated. After obtaining the image, the electromagnetic brake 57 is released, whereby the movable holding plate 51 moves in a direction of releasing compression on the breast 10.

According to the above compressing and holding operation, the subject person does not move when the breast is compressed and held, hence burden on the subject person is decreased. It is also possible to accurately position the breast with respect to the holding plate. By compressing and holding the breast, the projected sectional area increases and a good image can be obtained. Therefore the configuration of this example can easily implement accurate positioning of the holding plate and object (breast), without any burden on the subject person. Since the probe and the optical system on the probe side are fixed with respect to the housing, positional accuracy of these components can be guaranteed.

The measuring apparatus of the above mentioned example has the optical system which guides the light from the light source to both the movable holding plate and the fixed holding plate, and performs double irradiation. The configuration of the optical system, however, is not limited to this, but an optical system may be installed only for one of the movable holding plate and the fixed holding plate. In this case, light is irradiated from the one where the optical system is installed and not from the other, that is, a single irradiation configuration. The probe can also be installed in at least one of the holding plates. In this case, it is preferable to install both the probe and the optical system in the fixed holding plate, then the positional accuracy of the probe and the optical system can be maintained since the fixed holding plate is secured in the frame. If the probe and the optical system are installed in different holding plates, on the other hand, the probe and the optical system can be disposed in opposite positions interposing the breast of the subject person, hence a photoacoustic wave can be detected at a location close to the photoacoustic wave generation area.

### Example 2

According to a configuration of a measuring apparatus according to Example 2, a subject person bends forward and the upper part of the body is supported by a supporting member, and a breast 10 is held by a movable first holding member and a movable second holding member. Description on the members and the configurations, the same as Example 1, is omitted.

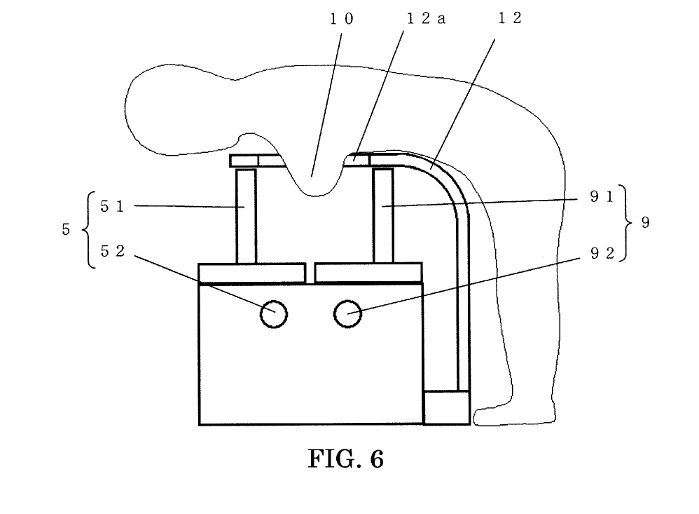

FIG. 6 is a side view of the supporting member and the holding mechanism of the measuring apparatus according to Example 2. The supporting member 12 of the measuring apparatus has an opening 12a for inserting a breast. The measuring apparatus has a first holding mechanism 5 and a second holding mechanism 9. The first holding mechanism 5 has a first holding member 51 for holding the breast 10 and a compression handle 52 for adjusting a position of the first holding member 51, just like Example 1. The second holding mechanism 9 also has a similar configuration as the first holding mechanism 5, and has a second holding member 91 for holding the breast 10, and a compression handle 92 for adjusting the position of the second holding member 91.

The first holding member 51 and the second holding member 91 linearly move in the normal line direction of the holding plate 51 by a linear guide, which is not illustrated, and compress and hold the breast 10. According to Example 2, the first holding member 51 and the second holding member 91 can move in a direction to compress the breast 10. Since the first holding member 51 and the second holding member 91 can relatively move with respect to the breast 10, the first holding member or the second holding member can be positioned for the breast 10, and the breast can be compressed and held by the first holding member and the second holding member. In the configuration of this example, an image is obtained in a state of the subject person standing while the body is supported by the supporting member 12. Therefore the supporting member is fixed in order to maintain stability for the subject person.

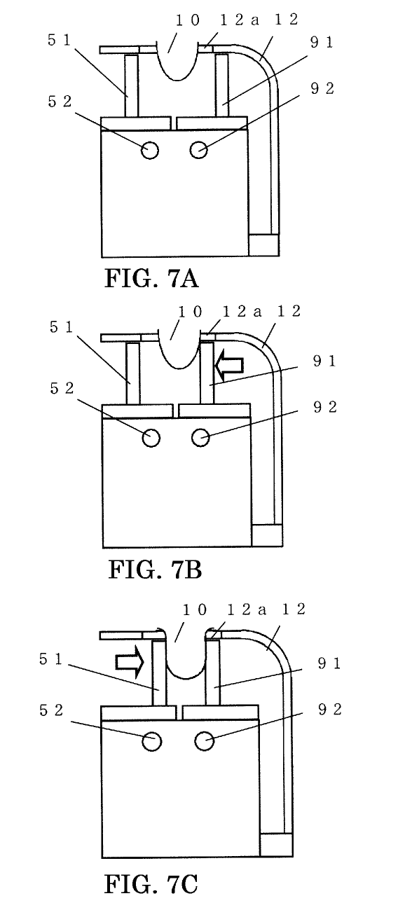

FIG. 7 are schematics depicting the positional relationships of the breast 10, the supporting member 12, the first holding member 51 and the second holding member 91 from inserting the breast into the opening 12a to compressing and holding the breast 10. FIG. 7A shows a state when the breast 10 is inserted into the opening 12a. FIG. 7B shows a state when a lower portion of the breast is contacted with the second holding member 91. FIG. 7C shows a state when an upper portion of the breast is compressed and held using the first holding member 51.

The subject person bends forward and places the upper part of their body on the supporting member 12. At this time, a breast of which an image is obtained is inserted into the opening 12a (FIG. 7A). Then the second holding member 91, on the lower portion of the breast side, is moved to contact the lower portion of the breast, and the second holding member 91 is locked by a switch disposed in the same manner as Example 1 (FIG. 7B). Then the first holding member 51 is moved to compress and hold the breast 10, and the first holding member 51 is locked by a switch disposed in the same manner as Example 1 (FIG. 7C).

According to the above compressing and holding operation, the subject person does not move when the breast is compressed and held, hence the burden on the subject person is decreased. It is also possible to accurately position the breast with respect to the holding plate by adjusting the moving distance of the holding plates. By compressing and holding the breast, the projected sectional area increases, and a good image can be obtained. Therefore according to this example, the positioning of the holding plates and the object (breast) can be performed accurately and easily while stably supporting the subject person, even if the subject person is standing.

### <Variant Form>

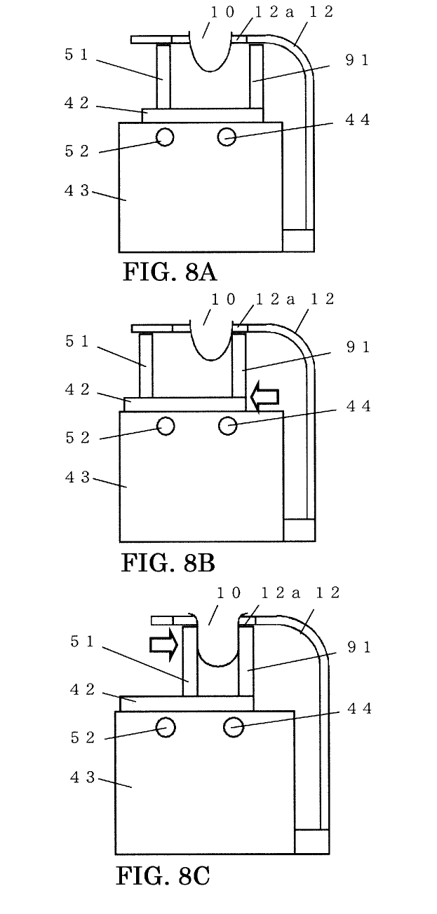

A variant form of Example 2 will now be described with reference to FIG. 8. Differences of this variant form from Example 2 are a configuration of the holding plates and a method of compressing and holding the breast. FIG. 8A shows a state when the breast 10 is inserted into the opening 12a. FIG. 8B shows a state when a lower portion of the breast is contacted with the second holding member 91. FIG. 8C shows a state when an upper portion of the breast is compressed and held using the first holding member 51. In FIG. 8, the first holding mechanism 5 including the first holding member 51 and the second holding member 91 are installed on a same base 42. The first holding member 51 is movable with respect to the base 42, and the second holding member 91 is fixed with respect to the base 42. The base 42 can be moved from the housing 43 in the normal line direction of the second holding member 91.

The position of the first holding member 51 can be adjusted by the handle 52, and the position of the base 42 can be adjusted by the handle 44. In the configuration of this variant form as well, the first holding member 51 and the second holding member 91 are movable, and the supporting member 12, the first holding member 51 and the second holding member 91 can be relatively moved in a direction of compressing the breast 10. First the breast 10 is inserted into the opening 12a (FIG. 8A). Then the second holding member 91 is contacted with a lower portion of the breast by adjusting the position of the base 42 using the handle (FIG. 8B). Then the position of the first holding member 51 is adjusted using the handle 52 (FIG. 8C). Thereby the breast 10 can be compressed and held.

Therefore according to the configuration of the variant form as well, the positioning of the holding plates and the object (breast) can be performed accurately and easily while decreasing the burden on the subject person, just like Example 2.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-098712, filed on Apr. 22, 2010 and Japanese Patent Application No. 2011-052069, filed on Mar. 9, 2011, which are hereby incorporated by reference herein in its entirety.

1. A measuring apparatus comprising:
a supporting member which supports a subject person and has an opening for inserting an object that is a part of the subject person; and
a first holding member and a second holding member which interpose and compress the object when the object is inserted through said opening,
wherein at least two of said supporting member, said first holding member and said second holding member can move independently from one another in a direction of compressing the object.

2. The measuring apparatus according to claim 1, further comprising:
a light source which generates light to be irradiated onto the object;
an optical system which is installed at least on one side of said first holding member and said second holding member, and guides the light from said light source to the object; and
a probe which is installed at least on one side of said first holding member and said second holding member, and receives an acoustic wave generated from the object which has absorbed the light irradiated from said optical system.

3. The measuring apparatus according to claim 2, further comprising a housing,
wherein said supporting member is installed on said housing and can move independently from said housing,
wherein one of said first and second holding members is movable with respect to said housing and the other is fixed with respect to said housing, and
wherein, after the object makes contact with said fixed holding member by movement of said supporting member, the said movable holding member moves so that the object is compressed between said movable holding member and said fixed holding member.

4. The measuring apparatus according to claim 3, wherein said probe is installed on the side of said fixed holding member.

5. The measuring apparatus according to claim 3, wherein said optical system is installed on the side of said fixed holding member.

6. The measuring apparatus according to claim 1, wherein
said supporting member is fixed,
said first and second holding members can move independently from each other, and
said first and second holding members move so as to interpose and compress the object therebetween.
